(19) United States
(12) Patent Application Publication
(10) Pub. No.: US 2025/0160742 A1
(43) Pub. Date: May 22, 2025

(54) FEEDBACK APPARATUS, FEEDBACK METHOD, AND PROGRAM

(71) Applicant: NIPPON TELEGRAPH AND TELEPHONE CORPORATION, Tokyo (JP)

(72) Inventor: Masahiro FUJINO, Tokyo (JP)

(73) Assignee: NIPPON TELEGRAPH AND TELEPHONE CORPORATION, Tokyo (JP)

(21) Appl. No.: 18/836,316

(22) PCT Filed: Feb. 10, 2022
(86) PCT No.: PCT/JP2022/005289

§ 371 Date: Aug. 6, 2024

(51) Int. Cl. A61B 5/00; G16H 50/20; G16H 50/30

(52) U.S. Cl. CPC A61B 5/486; G16H 50/20; G16H 50/30

(57) ABSTRACT

A technique which helps users achieve mindfulness is provided. A feedback device includes a feature quantity calculation part which calculates a physical condition feature quantity from physical condition data, an index calculation part which calculates a physical condition index using the physical condition feature quantity, an estimation result generation part which generates physical condition estimation result using the physical condition feature quantity and the physical condition index, a feedback part which presents a predetermined stimulus to the user, as feedback, on the basis of the physical condition estimation result, and a user reaction acquisition part which acquires a user reaction indicating that the user has noticed the feedback and in which the feedback part presents the stimulus, as feedback, in the form of increasing strength in a stepwise manner during a period from when the user's physical condition estimation result shows an abnormality for a predetermined period until the user notices the feedback, and presents the stimulus, as feedback, in the form of increasing or decreasing the stimulation in a stepwise manner in accordance with the user's physical condition estimation result during a period from when the user notices the feedback until the user's physical condition estimation result returns to normal for a predetermined period.

### TECHNICAL FIELD

The present invention relates to a technique for assisting realization of mindfulness.

### BACKGROUND ART

Mindfulness is a state of being aware of the various experiences that occur in the present moment such as sensations, emotions, and thoughts. When confronting unpleasant sensations, emotions, or thoughts, it is known that it is better to remain aware of them and wait for them to subside naturally, rather than to intentionally suppress or control them. However, in many cases, as soon as we pay attention to an experience such as an emotion, we are strongly conscious of that experience and the experience is retained by our consciousness and transformed or deteriorates. Therefore, training is required to be able to achieve mindfulness. A typical example of this practice is a method for noticing the breathing naturally occurring within oneself in daily life. When we pay attention to our naturally occurring breathing, we become conscious of our breathing and our breathing becomes unnatural. However, it is possible to gradually become aware of our breathing naturally by continuing this practice. Furthermore, you can become aware of the experiences that occur within yourself such as sensations, emotions, and thoughts in your daily life. Here, in daily life, it is extremely difficult to pay attention to the breathing which occurs naturally within oneself and to be aware of it as it is without controlling it. Therefore, a feedback technique is required for assisting this training.

NPL 1 discloses a technique for feeding back one's own breathing condition. In the technique described in NPL 1, after feeding back the pattern of the user's breathing condition, by changing the pattern stepwise from a user's breathing condition to an ideal breathing condition, the user is guided to intentionally control his or her own breathing condition to be a specific breathing condition.

### CITATION LIST

### Non Patent Literature

[NPL 1] Hiroki URATANI, “Development and Evaluation of Breathing-guided Biofeedback Device for Learning Relaxation,” Biofeedback Research, Vol. 46, No. 2, pp. 101-106, 2019.

### SUMMARY OF INVENTION

### Technical Problem

In this way, an object of the technique of NPL 1 is to intentionally control one's own breathing condition to be a specific breathing condition by paying attention to external feedback information. On the other hand, an object of mindfulness training is to help a user pay attention to the user's own internal state and become aware of the user's natural breathing condition without controlling it.

Therefore, an object of the present invention is to provide a technique which supports realization of mindfulness by a user.

### Solution to Problem

An aspect of the present invention is a feedback device which includes: a feature quantity calculation part which calculates a feature quantity of a user's physical condition data (hereinafter referred to as “physical condition feature quantity”) from data regarding the user's physical condition (hereinafter referred to as physical condition data); an index calculation part which calculates an index of the user's physical condition (hereinafter referred to as a “physical condition index”) using the user's physical condition feature quantity; an estimation result generation part which generates an estimation result regarding the user's physical condition (hereinafter referred to as a “physical condition estimation result”) using the user's physical condition feature quantity and the user's physical condition index; a feedback part which presents a predetermined stimulus to the user as feedback on the basis of the user's physical condition estimation result; and a user reaction acquisition part which acquires a user reaction indicating that the user has noticed the feedback, wherein the feedback part presents the stimulus, as feedback, in the form of increasing strength in a stepwise manner during a period from when the user's physical condition estimation result shows an abnormality for a predetermined period until the user notices the feedback, and presents the stimulus, as feedback, in the form of increasing or decreasing the stimulation in a stepwise manner in accordance with the user's physical condition estimation result during a period from when the user notices the feedback until the user's physical condition estimation result returns to normal for a predetermined period.

### Advantageous Effects of Invention

According to the present invention, it is possible to support realization of mindfulness by a user.

### BRIEF DESCRIPTION OF DRAWINGS

FIG. 1 is a block diagram showing a configuration of a feedback device 100.

FIG. 2 is a flowchart for describing an operation of the feedback device 100.

FIG. 3 is a block diagram showing a configuration of a feedback device 200.

FIG. 4 is a flowchart for describing an operation of the feedback device 200.

FIG. 5 is a block diagram showing a configuration of a physical condition estimation model learning device 300.

FIG. 6 is a flowchart for describing an operation of the physical condition estimation model learning device 300.

FIG. 7 is a block diagram showing a configuration of a feedback device 400.

FIG. 8 is a flowchart for describing an operation of the feedback device 400.

FIG. 9 is a diagram showing an example of a functional configuration of a computer which implements each device in an embodiment of the present invention.

### DESCRIPTION OF EMBODIMENTS

Embodiments of the present invention will be described in detail below. Note that constituent elements having the same functions are denoted by the same reference numerals and redundant explanations thereof will be omitted.

### Technical Background

The design concept of a device in an embodiment of the present invention will be explained. The device in the embodiment of the present invention assists in achieving mindfulness. Here, the realization of mindfulness is the ability to pay attention to the experiences of sensations, emotions, and thoughts occurring within oneself and to be aware of them as they are without intentionally controlling them. Thus, physiological phenomena which can be used for mindfulness training are required to have the characteristics of being able to occur unconsciously and to be moved intentionally. Therefore, in the device according to the embodiment of the present invention, breathing is used as a physiological phenomenon having such characteristics.

The device in the embodiment of the present invention is needed to allow attention to be paid promptly to current breathing conditions when it deviates from the average range for breathing conditions in everyday life. Simple feedback, such as sounding an alarm when deviations are outside of the average range, draws attention to the feedback itself and can lead to overreliance on feedback. In addition, a person may start paying attention to external information such as alarms, making it difficult to pay attention to the state of breathing itself. Thus, in the device according to the embodiment of the present invention, stimuli such as sounds, vibrations, and lights linked to breathing are presented as feedback in a form in which they are increased in a stepwise manner to give awareness. This makes it possible to encourage the user to pay attention to the breathing condition without inhibiting the user from paying attention to the breathing condition itself.

Furthermore, the device in accordance with embodiment of the present invention is required for enhancing the ability to be aware of breathing conditions as it occurs. Thus, in the device according to the embodiment of the present invention, feedback is presented in a manner in which it is linked to the user's breathing condition so that the breathing condition is not controlled. In addition, feedback is presented in a form in which it becomes progressively stronger or weaker on the basis of a difference between the abnormal breathing condition and the normal breathing condition so that it is possible to understand experientially that an abnormal breathing condition is returning to a normal breathing condition just by noticing this.

Further, in order to assist in directing attention to the breathing condition itself, the device in an embodiment of the present invention allows the user to determine the level of stress and the type of emotion relating to the breathing condition. This makes it possible to encourage people to pay attention to their breathing condition and mental states.

### First Embodiment

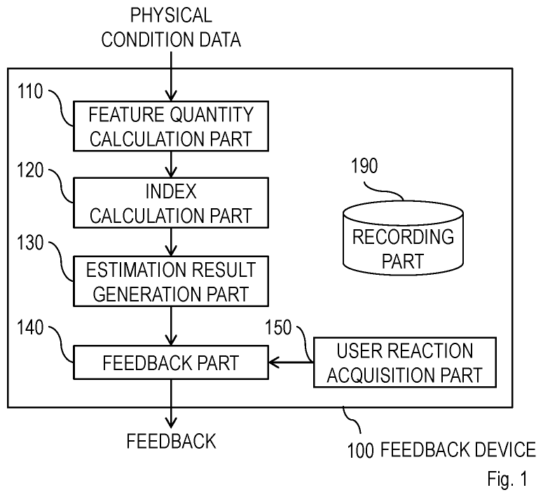

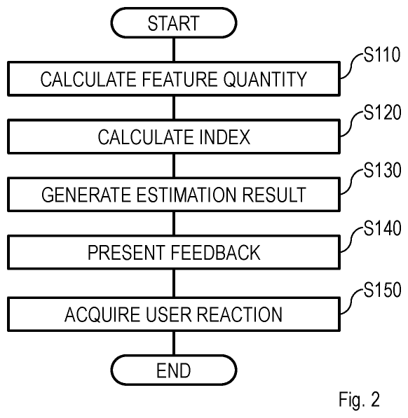

A feedback device 100 will be described below with reference to FIGS. 1 and 2. FIG. 1 is a block diagram showing a configuration of the feedback device 100. FIG. 2 is a flowchart for describing an operation of the feedback device 100. As shown in FIG. 1, the feedback device 100 includes a feature quantity calculation part 110, an index calculation part 120, an estimation result generation part 130, a feedback part 140, a user reaction acquisition part 150, and a recording part 190. The recording part 190 is a constituent element which appropriately records information necessary for processing by the feedback device 100.

An operation of the feedback device 100 will be explained with reference to FIG. 2.

In S110, the feature quantity calculation part 110 receives as input data relating to the user's physical condition (hereinafter referred to as “physical condition data”), and calculates and outputs a feature quantity of the user's physical condition data (hereinafter referred to as a “physical condition feature quantity”) from the physical condition data. An example of a physical condition is a breathing condition. In this case, the physical condition data is, for example, data relating to the breathing condition in daily life acquired using a portable breathing measurement device. Portable breathing measurement devices include, for example, a breathing measurement band, a measurement device having a millimeter wave radar installed therein, a breathing flow measurement mask, a measurement device having a thermometer installed in a general mask installed therein, and a device which measures breathing sounds. When the physical condition is a breathing condition, the feature quantity calculation part 110 calculates, for example, the number of breathings, the breathing rate, the breathing amount, the breathing rhythm, and the breathing pattern as the physical condition feature quantities.

Note that the feature quantity calculation part 110 calculates the physical condition feature quantity at any time while the physical condition data is being acquired.

In S120, the index calculation part 120 receives, as inputs, the physical condition feature quantity calculated in S110, and calculates and outputs an index of the user's physical condition (hereinafter referred to as a physical condition index) using the physical condition feature quantity. When the physical condition is a breathing condition, the index calculation part 120 calculates the average range of the breathing condition in daily life as a physical condition index.

Note that the index calculation part 120 calculates the physical condition index at any time while the physical condition data is being acquired.

In S130, the estimation result generation part 130 receives, as inputs, the physical condition feature quantity calculated in S110 and the physical condition index calculated in S120, and generates and outputs an estimation result (hereinafter referred to as “physical condition estimation results”) regarding the user's physical condition using the physical condition feature quantity and the physical condition index. The physical condition estimation result includes, for example, whether the physical condition is normal or abnormal and the degree of deviation of the physical condition feature quantity from the physical condition index. The estimation result generation part 130 generates a physical condition estimation result indicating abnormality when the physical condition feature quantity deviates from the physical condition index for a predetermined period, and otherwise generates a physical condition estimation result indicating normality.

Note that the estimation result generation part 130 generates the physical condition estimation result at any time while the physical condition data is being acquired.

In S140, the feedback part 140 inputs the physical condition estimation result generated in S130 and presents a predetermined stimulus to the user as feedback on the basis of the physical condition estimation result. The feedback part 140 starts presenting stimulation as feedback when the user's physical condition estimation result shows an abnormality for a predetermined period of time. Also, the feedback part 140 presents feedback in the form of increasing stimulation stepwise from when the user's physical condition estimation result shows an abnormality for a predetermined period until the user notices the feedback. Furthermore, the feedback part 140 presents feedback in the form of increasing or decreasing the stimulation in a stepwise manner in accordance with the user's physical condition estimation result from the time the user notices the feedback until the user's physical condition estimation result returns to normal for a predetermined period. The feedback part 140 ends presenting the stimulation as feedback when the user's physical condition estimation result returns to normal for a predetermined period. Alternatively, the feedback part 140 may end presenting the stimulus as feedback when the user takes deep breaths a predetermined number of times (for example, three times). While presenting the stimulus as feedback, the feedback part 140 may gradually increase or decrease the stimulus in a stepwise manner in accordance with the extent to which the physical condition feature quantity deviates from the physical condition index. Note that the feedback part 140 can use sound, vibration and light as stimuli. In this case, as a device which presents stimulation as feedback to the user, for example, earphones which do not cover the ears, bone conduction earphones, a smart watch with a built-in vibrator, or an LED light can be used.

In S150, the user reaction acquisition part 150 acquires and outputs a user reaction indicating that the user noticed the feedback presented in S140. When the user notices the feedback, the user inputs a user reaction using, for example, a smartphone or smart watch. In this case, it is a good idea to have the user's reaction output by simply pressing a button on the smartphone or the smartwatch. Alternatively, if the physical condition is a breathing condition, a user reaction indicating that the user has noticed it may be automatically input by detecting deep breathing with the portable breathing measurement device used to obtain the physical condition data.

According to the embodiment of the present invention, it is possible to support the realization of mindfulness by the user. When the physical condition is a breathing condition, it is possible to pay attention to one's own breathing condition by giving feedback based on the breathing condition. In addition, it becomes possible to accept the state of breathing as it is without controlling it by presenting feedback in a form linked to the breathing condition and giving feedback in a form in which it is increased or decreased in a stepwise manner on the basis of the difference between the abnormal breathing condition and the normal breathing condition.

### Second Embodiment

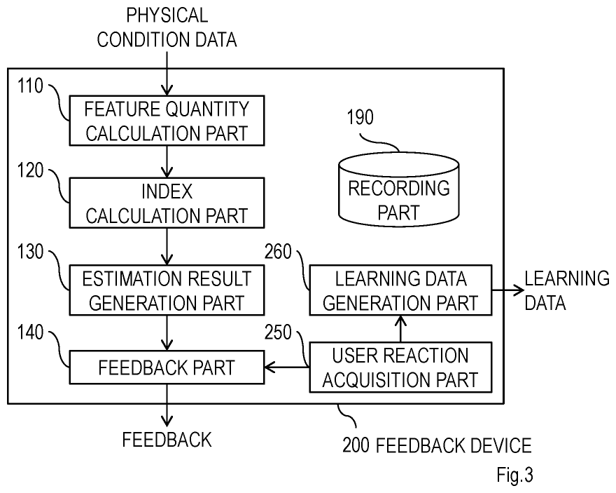

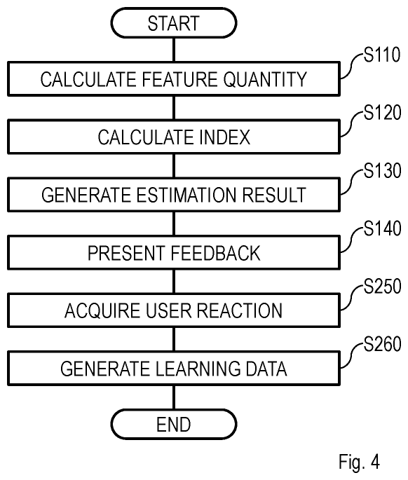

A feedback device 200 will be described below with reference to FIGS. 3 and 4. FIG. 3 is a block diagram showing a configuration of the feedback device 200. FIG. 4 is a flowchart for describing an operation of the feedback device 200. As shown in FIG. 3, the feedback device 200 includes a feature quantity calculation part 110, an index calculation part 120, an estimation result generation part 130, a feedback part 140, a user reaction acquisition part 250, a learning data generation part 260, and a recording part 190. The recording part 190 is a constituent element which appropriately records information necessary for processing of the feedback device 200. That is to say, the feedback device 200 differs from the feedback device 100 in that it includes a user reaction acquisition part 250 instead of the user reaction acquisition part 150 and that it further includes a learning data generation part 260.

An operation of the feedback device 200 will be explained with reference to FIGS. 4. S250 and S260 will be described below.

In S250, the user reaction acquisition part 250 acquires and outputs the user's reaction to the feedback presented in S140 (hereinafter referred to as “user reaction”). Some examples of user reactions are described below.
(1) When the user reaction is an evaluation by the user regarding his or her own state of mind (hereinafter referred to as “subjective evaluation”)

The state of mind refers to, for example, the stress level and the type of emotion. In this case, after the user notices the feedback, the user uses, for example, a smartphone or a smartwatch to observe his/her own breathing condition and ascertain his or her own state of mind and inputs his/her own state of mind as a user reaction. For example, one of the predetermined options may be selected by simply pressing a button on a smartphone or smartwatch, and a subjective evaluation may be output as a user reaction.
(2) When the user reaction is an indication that the user noticed the feedback

In this case, the user may input using the same method as the user reaction acquisition part 150.
(3) When the user reaction is a combination of the user's subjective evaluation and the indication that the user noticed the feedback

In this case, the user may input using the same method as (1) and (2).

In S260, the learning data generation part 260 receives, as inputs, the physical condition feature quantity calculated in S110 and the user reaction acquired in S250, generates, as learning data, a set of the user's physical condition feature quantity and teacher data, which have the user reaction or a value calculated from the user reaction as the teacher data, from the physical condition feature quantity and the user reaction, and outputs the set. When the user reaction is a subjective evaluation by the user, the subjective evaluation is used as teacher data. Also, when the user reaction is an indication that the user noticed the feedback, the interval between the time when the feedback starts and the time when the user notices the feedback and the interval between the time when the user notices the feedback and the time when the feedback ends are used as teacher data. These intervals indicate the user's degree of mindfulness realization. Furthermore, when the user reaction is a combination of the user's subjective evaluation and the indication that the user noticed the feedback, a set of the subjective evaluation and the interval between the time when the feedback starts and the time when the user notices the feedback and a set of the subjective evaluation and the interval between the time when the user notices the feedback and the time when the feedback ends are used as teacher data. These intervals indicate the degree of realization of mindfulness for stress level of the user and the degree of realization of mindfulness for type of emotion of the user.

According to the embodiment of the present invention, it is possible to support the realization of mindfulness by the user. When the physical condition is breathing condition, it is possible to pay attention to one's breathing condition by giving feedback based on a breathing condition. In addition, it becomes possible to accept the state of breathing as it is without controlling it by presenting feedback in a form linked to the breathing condition and giving feedback in a form in which it is increased or decreased in a stepwise manner on the basis of the difference between the abnormal breathing condition and the normal breathing condition. Furthermore, when the user reaction is a subjective evaluation by the user, it becomes possible to pay attention to the state of mind by determining a stress level and types of emotions relating to the breathing condition for himself/herself.

### Third Embodiment

A physical condition estimation model learning device 300 uses the learning data generated by a feedback device 200 to learn a physical condition estimation model. Here, the physical condition estimation model is a function which inputs the user's physical condition feature quantity and outputs an evaluation value regarding the user. Also, the evaluation value regarding the user is an evaluation value regarding the user's state of mind and an evaluation value regarding the degree of realization of mindfulness of the user indicated by the reaction speed, the feedback end speed, the reaction speed for stress level of the user, the reaction speed for type of emotion of the user, the feedback end speed for stress level of the user and the feedback end speed for type of emotion of the user. Note that the physical condition estimation model can be configured as, for example, a deep neural network.

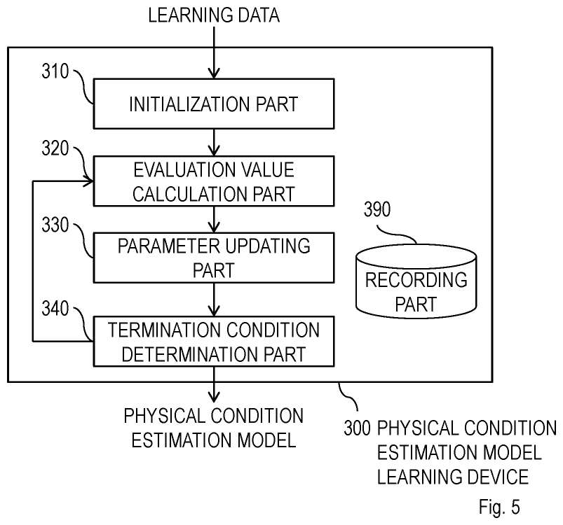

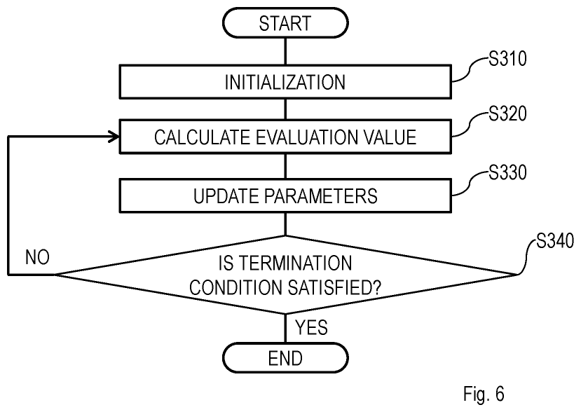

The physical condition estimation model learning device 300 will be described below with reference to FIGS. 5 and 6. FIG. 5 is a block diagram showing a configuration of the physical condition estimation model learning device 300. FIG. 6 is a flowchart for describing an operation of the physical condition estimation model learning device 300. As shown in FIG. 5, the physical condition estimation model learning device 300 includes an initialization part 310, an evaluation value calculation part 320, a parameter updating part 330, a termination condition determination part 340, and a recording part 390. The recording part 390 is a constituent element which appropriately records information necessary for the processing of the physical condition estimation model learning device 300. The recording part 390 may record the learning data generated by the feedback device 200 in advance. Moreover, the recording part 390 may record in advance the initial values of the parameters of the physical condition estimation model.

An operation of the physical condition estimation model learning device 300 will be described with reference to FIG. 6.

In S310, the initialization part 310 initializes the parameters of the physical condition estimation model. The initialization part 310 may initialize the parameters of the physical condition estimation model using initial values recorded in advance in the recording part 390 and initialize the parameters of the physical condition estimation model using initial values generated using random numbers.

In S320, the evaluation value calculation part 320 receives, as an input, the user's physical condition feature quantity which is an element of the learning data and calculates and outputs the evaluation value regarding the user using the physical condition feature quantity. When calculating the evaluation value, the evaluation value calculation part 320 uses the parameters which are being learned as the parameters of the neural network which constitutes the physical condition estimation model.

When the teacher data is subjective evaluation by the user, the evaluation value regarding the user is the evaluation value regarding the state of mind of the user. For example, when the state of mind is in a state of stress, the evaluation values calculated by the evaluation value calculation part 320 are, for example, a value representing strong stress, a value representing medium stress, and a value representing weak stress. Also, for example, when the state of mind is a type of emotion, the evaluation value calculated by the evaluation value calculation part 320 includes, for example, a value representing a pleasant emotion, a value representing an unpleasant emotion, a value representing an emotion that is neither pleasant nor unpleasant, a value representing a joyful emotion, a value representing an angry emotion, a value representing a disgusting emotion, a value representing a fear emotion, a value representing a happy emotion, a value representing a sad emotion, a value representing a surprise emotion, and a value representing a neutral emotion.

In addition, when the teacher data is either the interval between the time the feedback starts and the time the user notices the feedback, or the interval between the time the user notices the feedback and the end of the feedback, the evaluation value regarding the user is an evaluation value regarding the degree of realization of mindfulness by the user. When the teacher data is the interval between when the user starts feedback and when the user notices the feedback, the evaluation value calculated by the evaluation value calculation part 320 includes, for example, a value which indicates rapid awareness (that is, a value which indicates that the prerequisites for mindfulness are successfully realized), a value which indicates a moderate level of awareness (that is, a value which indicates that the prerequisites for mindfulness have been achieved to some extent), and a value which indicates slow awareness (that is, a value which indicates that the prerequisites for mindfulness is poorly realized). Furthermore, when the teacher data is a set of the subjective evaluation by the user and the interval between the feedback start time and the time when the user noticed the feedback or a set of the subjective evaluation and the interval between the time when the user noticed the feedback and the feedback end time, the evaluation value regarding the user is an evaluation value regarding the degree of realization of mindfulness for stress level of the user and the degree of realization of mindfulness for type of emotion of the user. In addition, when the teacher data is the interval between when the user notices the feedback and when the feedback ends, the evaluation value calculated by the evaluation value calculation part 320 includes, for example, a value which indicates that convergence is fast (that is, a value which indicates that mindfulness is successfully achieved), a value which indicates moderate convergence (that is, a value which indicates that mindfulness has been achieved to some extent), and a value which indicates slow convergence (that is, a value which indicates poor mindfulness). Furthermore, when the teacher data is a set of the user's subjective evaluation and the interval between the time when the feedback starts and the time when the user notices the feedback or a set of the user's subjective evaluation and the interval between the time when the user notices the feedback and the time when the feedback ends, the evaluation value regarding the user is an evaluation value regarding the degree of realization of mindfulness for stress level of the user and the degree of realization of mindfulness for type of emotion of the user.

In S330, the parameter updating part 330 updates and outputs the parameters of the physical condition estimation model using the evaluation value calculated in S320 and the teacher data that is an element of the learning data input in S320.

In S340, the termination condition determination part 340 determines whether or not a termination condition regarding parameter update which has been set in advance is satisfied, and, when the termination condition is satisfied, outputs the parameters updated in S330 and the process ends. On the other hand, the termination condition determination part 340 repeatedly performs the processes of S320 to S330 when the termination condition is not satisfied. The termination condition may be, for example, whether the number of times the processes in S320 to S330 have been performed has reached a predetermined number (for example, 5000 times).

According to the embodiment of the present invention, it becomes possible to learn the model in which the evaluation values regarding the user's state of mind, the evaluation values regarding the user's degree of mindfulness realization, the evaluation values regarding the user's degree of mindfulness realization for stress level, and the evaluation values regarding the user's degree of mindfulness realization for type of emotion are output on the basis of the user's physical condition.

### Fourth Embodiment

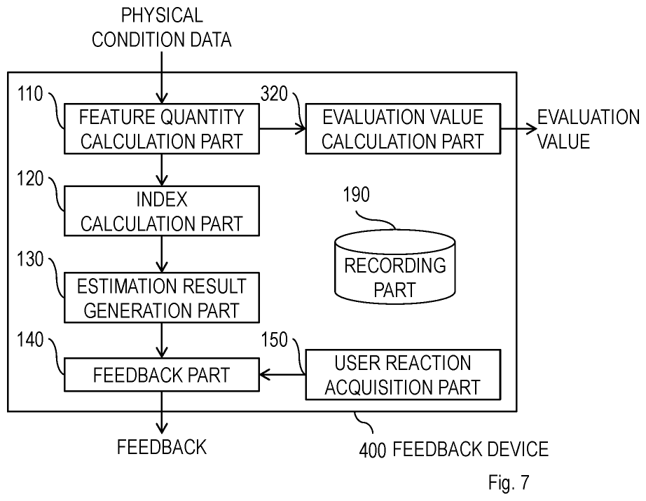

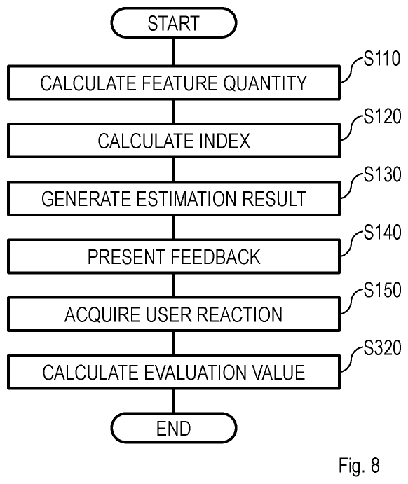

A feedback device 400 will be described below with reference to FIGS. 7 and 8. FIG. 7 is a block diagram showing a configuration of the feedback device 400. FIG. 8 is a flowchart for describing an operation of the feedback device 400. As shown in FIG. 7, a feedback device 400 includes a feature quantity calculation part 110, an index calculation part 120, an estimation result generation part 130, a feedback part 140, a user reaction acquisition part 150, an evaluation value calculation part 320, and a recording part 190. The recording part 190 is a constituent element which appropriately records information necessary for the processing of the feedback device 400. That is to say, the feedback device 400 differs from the feedback device 100 in that it further includes evaluation value calculation part 320.

An operation of the feedback device 400 will be explained with reference to FIG. 8. S320 will be explained below.

In S320, the evaluation value calculation part 320 receives, as an input, the user's physical condition feature quantity calculated in S110 and calculates and outputs the evaluation value regarding the user using the physical condition feature quantity. When calculating the evaluation value, the evaluation value calculation part 320 uses the parameters learned using the physical condition estimation model learning device 300 as the parameters of the neural network which constitutes the physical condition estimation model.

According to the embodiment of the present invention, it is possible to support the realization of mindfulness by the user. When the physical condition is breathing, it is possible to pay attention to one's breathing condition by giving feedback based on a breathing condition and it becomes possible to accept the breathing condition as it is without controlling it by presenting feedback in a form linked to the breathing condition and giving feedback in a form in which it is increased or decreased in a stepwise manner on the basis of the difference between the abnormal breathing condition and the normal breathing condition. Furthermore, it is possible to support the realization of mindfulness using an evaluation value regarding the user's state of mind, an evaluation value for the degree of realization of mindfulness of the user, an evaluation value regarding the degree of mindfulness realization for stress level of the user, or an evaluation value regarding the degree of mindfulness realization for type of emotion of the user, which are based on the user's physical condition.

### Application Example

In the above explanation, breathing is used as a physiological phenomenon for understanding the physical condition. As explained in <Technical background>, physiological phenomena which can be used for realizing mindfulness are physiological phenomena which occur unconsciously and can be manipulated intentionally and any physiological phenomenon can be used as long as it has such characteristics. For example, a tense state, heartbeat, blinking, balance, posture, and poor posture can be used as physiological phenomena to understand the physical condition.

### Supplementary Note

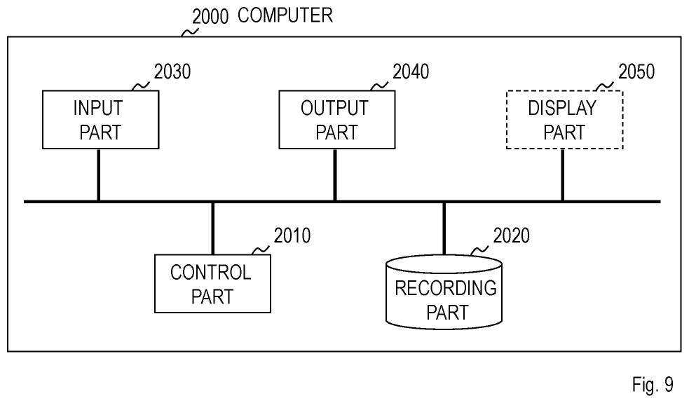

FIG. 9 is a diagram showing an example of a functional configuration of a computer 2000 which implements each of the above-described devices. The processing in each of the above-described devices can be carried out by causing the recording part 2020 to read a program for causing the computer 2000 to function as each of the above-described devices and operating a control part 2010, an input part 2030, an output part 2040, and the like.

The device of the present invention has, for example, as single hardware entities, an input part to which a keyboard and the like can be connected, an output part which can be connected to a liquid crystal display and the like, a communication part to which a communication device (for example, a communication cable) capable of communicating with the outside of the hardware entity can be connected, a CPU (Central Processing Unit, which may include cache memory, registers and the like), a RAM and a ROM, which are memories, external storage devices such as hard disks, and a bus which connects input parts, output parts, communication parts, the CPU, RAM, ROM, and external storage devices thereof so that data can be exchanged between them. Furthermore, if necessary, the hardware entity may include a device (drive) which can read and write a recording medium such as a CD-ROM. A physical entity including such hardware resources includes a general-purpose computer.

The external storage device of the hardware entity stores a program necessary for realizing the above-described functions, data necessary for processing this program, and the like (the present invention is not limited to external storage devices; for example, programs may be stored in a ROM that is a read-only storage device). Furthermore, data obtained through processing of these programs is appropriately stored in a RAM, an external storage device, or the like.

In the hardware entity, each program stored in an external storage device (or a ROM or the like) and the data necessary for processing each program are read into a memory as needed and interpreted, executed, and processed using the CPU as appropriate. As a result, the CPU realizes a predetermined function (each constituent element represented by “part” or “unit” and the like described above).

The present invention is not limited to the above-described embodiments and can be modified as appropriate without departing from the spirit of the present invention. Furthermore, the processes described in the above embodiments may not only be performed in chronological order according to the order described, but may also be perform in parallel or individually depending on the processing capacity of the device that executes the processes or as necessary.

As described above, when the processing functions of the hardware entity (device of the present invention) described in the above embodiments are implemented by a computer, the processing contents of the functions included in the hardware entity are described using a program. Also, the processing functions of the hardware entity described above are realized on the computer by executing this program on a computer.

A program describing the contents of this process can be recorded on a computer-readable recording medium. The computer-readable recording medium may be of any type such as a magnetic recording device, an optical disk, a magneto-optical recording medium or a semiconductor memory.

Specifically, for example, a hard disk device, a flexible disk, a magnetic tape, or the like can be used as the magnetic recording device, a DVD (Digital Versatile Disc), DVD-RAM (Random Access Memory), a CD-ROM (Compact Disc Read Only Memory), CD-R (Recordable)/RW (ReWritable), and the like can be used as the optical disc, an MO (Magneto-Optical disc) or the like can be used as the magneto-optical recording medium, and an EEP-ROM (Electronically Erasable and Programmable-Read Only Memory) or the like can be used as the semiconductor memory.

Also, this program will be distributed, for example, by selling, transferring, and lending portable recording media such as DVDs and CD-ROMs recording the program. Furthermore, this program may be distributed by storing the program in the storage device of the server computer and transferring the program from the server computer to another computer over a network.

A computer which executes such a program, for example, first stores a program recorded on a portable recording medium or a program transferred from a server computer in its own storage device. Also, when performing the process, this computer reads the program stored in its own storage device and executes the process according to the read program. Furthermore, another form of execution of this program is for the computer to directly read the program from a portable recording medium and execute the process according to the program and each time a program is transferred from the server computer to this computer, processes according to the received program may be executed one after another. Furthermore, it is also possible to perform the above processing using a so-called ASP (Application Service Provider) type service, which does not transfer programs from the server computer to this computer, but only implements processing functions by issuing execution instructions and obtaining results. Note that the program in this embodiment includes information which is used for processing by an electronic computer and is equivalent to a program (data which is not a direct command to the computer but has the property of regulating computer processing or the like).

Furthermore, although a hardware entity is configured by executing a predetermined program on a computer in this embodiment, at least part of these processing contents may be implemented by hardware.

The foregoing descriptions of embodiments of the invention have been presented for purposes of illustration and description. There is no intent to be exhaustive or to limit the invention to the precise form disclosed. Modifications and variations are possible in light of the above teachings. The embodiments have been chosen and expressed to provide the best illustration of the principles of the invention and to enable those skilled in the art to utilize the present invention in various embodiments and with various modifications as suitable for the practical use contemplated. All such modifications and variations are within the scope of the invention as defined by the appended claims, interpreted in accordance with the breadth to which they are fairly and legally entitled.

1. A feedback device, comprising:
a feature quantity calculation circuitry which calculates a feature quantity of a user's physical condition data (hereinafter referred to as “physical condition feature quantity”) from data regarding the user's physical condition (hereinafter referred to as physical condition data); an index calculation circuitry which calculates an index of the user's physical condition (hereinafter referred to as a “physical condition index”) using the user's physical condition feature quantity;
an estimation result generation circuitry which generates an estimation result regarding the user's physical condition (hereinafter referred to as a “physical condition estimation result”) using the user's physical condition feature quantity and the user's physical condition index;
a feedback circuitry which presents a predetermined stimulus to the user as feedback on the basis of the user's physical condition estimation result; and
a user reaction acquisition circuitry which acquires a user reaction indicating that the user has noticed the feedback,
wherein the feedback circuitry
presents the stimulus, as feedback, in the form of increasing strength in a stepwise manner during a period from when the user's physical condition estimation result shows an abnormality for a predetermined period until the user notices the feedback, and presents the stimulus, as feedback, in the form of increasing or decreasing the stimulation in a stepwise manner in accordance with the user's physical condition estimation result during a period from when the user notices the feedback until the user's physical condition estimation result returns to normal for a predetermined period.

2. A feedback device, comprising:
a feature quantity calculation circuitry which calculates a feature quantity of a user's physical condition data (hereinafter referred to as physical condition feature quantity) from data regarding the user's physical condition (hereinafter referred to as “physical condition data”); an index calculation circuitry which calculates an index of the user's physical condition (hereinafter referred to as a “physical condition index”) using the user's physical condition feature quantity;
an estimation result generation circuitry which generates an estimation result regarding the user's physical condition (hereinafter referred to as “physical condition estimation result”) using the user's physical condition feature quantity and the user's physical condition index;
a feedback circuitry which presents a predetermined stimulus to the user as feedback on the basis of the user's physical condition estimation result;
a user reaction acquisition circuitry which acquires the user's reaction to the feedback (hereinafter referred to as a “user reaction”); and
a learning data generation circuitry which generates, as learning data, a set of the user's physical condition feature quantity and teacher data, which have the user reaction or a value calculated from the user reaction as the teacher data, from the user's physical condition feature quantity and the user reaction.

3. The feedback device according to claim 2, wherein
the user reaction is either an evaluation by the user regarding his or her own state of mind (hereinafter referred to as “subjective evaluation”), an indication that the user has noticed the feedback, or a combination of a subjective evaluation and an indication that the user has noticed the feedback.

4. The feedback device according to claim 1, wherein
the physical condition data is data relating to a breathing condition.

5. A feedback method, comprising:
a feature quantity calculation step of calculating, by a feedback device, a feature quantity of a user's physical condition data (hereinafter referred to as “physical condition feature quantity) from data relating to the user's physical condition (hereinafter referred to as “physical condition data”);
an index calculation step of calculating, by the feedback device, an index of the user's physical condition (hereinafter referred to as a “physical condition index”) using the user's physical condition feature quantity;
an estimation result generation step of generating, by the feedback device, an estimation result regarding the user's physical condition (hereinafter referred to as a “physical condition estimation result”) using the user's physical condition feature quantity and the user's physical condition index;
a feedback step of presenting, by the feedback device, a predetermined stimulus to the user, as feedback, on the basis of the user's physical condition estimation result; and
a user reaction acquisition step of acquiring, by the feedback device, a user reaction indicating that the user has noticed the feedback,
wherein the feedback step presents the stimulus, as feedback, in the form of increasing strength in a stepwise manner during a period from when the user's physical condition estimation result shows an abnormality for a predetermined period until the user notices the feedback, and presents the stimulus, as feedback, in the form of increasing or decreasing the stimulation in a stepwise manner in accordance with the user's physical condition estimation result during a period from when the user notices the feedback until the user's physical condition estimation result returns to normal for a predetermined period.

6. A non-transitory computer-readable storage medium which stores a program causing a computer to function as the feedback device according to claim 1.

7. The feedback device according to claim 2, wherein
the physical condition data is data relating to a breathing condition.
